(19) United States
(12) Patent Application Publication
(10) Pub. No.: US 2025/0152758 A1
(43) Pub. Date: May 15, 2025

(54) PROBE FOR NUCLEAR MEDICAL TESTING

(71) Applicant: The University of Tokyo, Tokyo (JP)

(72) Inventors: Yasuteru URANO, Tokyo (JP); Mako KAMIYA, Tokyo (JP); Atsuki ABE, Tokyo (JP); Hirofumi FUJII, Tokyo (JP); Kazunobu OHNUKI, Tokyo (JP)

(73) Assignee: The University of Tokyo, Tokyo (JP)

(21) Appl. No.: 18/834,373

(22) PCT Filed: Jan. 31, 2023
(86) PCT No.: PCT/JP2023/003072

§ 371 Date: Jul. 30, 2024

Related U.S. Application Data
(60) Provisional application No. 63/304,708, filed on Jan. 31, 2022.

(51) Int. Cl. A61K 51/04; C07C 233/05; C07D 207/16; C07H 3/02; C07H 15/04

(52) U.S. Cl. CPC A61K 51/0491; A61K 51/0446; C07C 233/05; C07D 207/16; C07H 3/02; C07H 15/04; A61K 2123/00

(57) ABSTRACT

Provided is a novel compound that is promising as a probe for nuclear medical examination. The compound is represented by the following general formula (I) or a salt thereof.

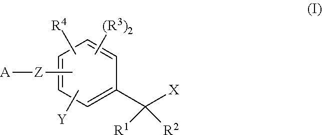

### TECHNICAL FIELD

The present invention relates to a novel compound that is promising as a probe for nuclear medical examination, and a pharmaceutical composition used in nuclear medical examination using the compound.

### BACKGROUND ART

The present inventors have hitherto found that peptidase activity such as γ-glutamyl transpeptidase (GGT) is cancer-specifically enhanced by fluorescence imaging using living cells and clinical specimens, and have succeeded in rapidly visualizing a small disseminated tumor by topically spraying a fluorescent probe having a γ-glutamyl group (gGlu-HMRG) or the like (Non Patent Literature 1).

However, cancer diagnosis by fluorescence imaging has an advantage that rapid diagnosis is possible due to its high temporal resolution, but it is difficult to detect cancer in a deep part due to low tissue permeability of visible light.

On the other hand, nuclear medical examination such as scintigraphy, SPECT (single-photon emission computed tomography), and PET (positron emission tomography) can be used for functional diagnosis of a deep part of a living body, and these studies have been actively conducted in recent years. These nuclear medical examinations are methods of administering a medicament (drug) containing a radionuclide to a patient and measuring radiation emitted from the radionuclide contained in the medicament (drug) localized in a target tissue or target organ of the patient to examine the presence of a tumor or the like in the target tissue or target organ. As such a medicament, a compound having a radionuclide such as iodine (¹²³I) is used, but there are few development examples of an effective radioisotope (RI) tracer targeting cancer cell-specific hydrolase activity.

### CITATION LIST

### Non Patent Literature

Non Patent Literature 1: Urano, Y. et al. Sci. Transl. Med. 2011, 3 (110), 110-119

### SUMMARY OF INVENTION

### Technical Problem

An object of the present invention is to provide a novel compound that is promising as a probe for nuclear medical examination.

### Means to Solve the Problem

The fluorescent probe 4-CH₂F-HMDiEtR-gGlu developed by the laboratory of the present inventors reacted with GTT to produce a reactive azaquinone methide, which was attacked by intracellular nucleophiles to become fluorescent and self-immobilize, enabling durable tumor imaging against washout.

Based on these facts, the present inventors have incorporated quinone methide chemistry into molecular design, and developed a probe for low molecular weight nuclear medical examination using a new I-125 or the like as a labeled nuclide. Then, the present inventors have considered that the present probe is specifically hydrolyzed by an enzyme such as GGT to produce an azaquinone methide reactive intermediate which is an electrophilic species, and this forms a covalent bond with a protein or the like in a cell, whereby the probe is metabolically trapped, and can be accumulated in cancer at a high concentration, thereby completing the present invention.

That is, the present invention has the following configuration.

[1] A compound represented by the following general formula (I) or a salt thereof.

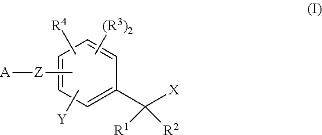

wherein:
X is selected from the group consisting of a fluorine atom, an ester group (—OC(═O)—R′), a carbonate group (—OCO₂—R′), a carbamate group (—OCONH—R′), phosphoric acid and its ester group (—OP(═O)(—OR′)(—OR″), and sulfuric acid and its ester group (—OSO₂—OR′),
wherein R′, R″ are each independently selected from a substituted or unsubstituted alkyl group or a substituted or unsubstituted aryl group;
Y is —NH—CO-L, —NH-L′, or —OL′,
wherein L is a partial structure of an amino acid,
L′ is a saccharide or a partial structure of a saccharide, a saccharide or a partial structure of a saccharide having a self-cleaving linker, or an amino acid or a peptide having a self-cleaving linker;
R¹ and R² are each independently selected from a hydrogen atom or a monovalent substituent;
R³ is a hydrogen atom or 1 to 2 identical or different monovalent substituents present on the benzene ring;
R⁴ is a hydrogen atom, or a substituent or molecule capable of altering pharmacokinetics,
the substituent or molecule may be bonded to the benzene ring via a linker;
Z represents a single bond or a linking group; and
A represents a radionuclide.

[2] The compound or a salt thereof according to [1], wherein the radionuclide is selected from the group consisting of ¹²⁵I, ²¹¹At, ¹⁸F, ¹⁵O, ¹²³I, ¹³¹I, ¹²⁴I, and ¹¹C.

[3] The compound or a salt thereof according to [1] or [2], wherein the substituent or molecule capable of altering pharmacokinetics is introduced into the benzene ring via a linker or directly.

[4] The compound or a salt thereof according to any one of [1] to [3], wherein the linker is selected from the group consisting of an alkylene group (with the proviso that one or more —CH₂— of the alkylene group may be replaced by —O—, —S—, —NH—, or —CO—.), arylene (including heteroarylene), cycloalkylene, an alkoxyl group, a polyethylene glycol chain, and a group formed by arbitrarily bonding two or more groups selected from these groups.

[5] The compound or a salt thereof according to any one of [1] to [4], wherein the linking group of Z is selected from the group consisting of an alkylene group (with the proviso that one or more —CH₂— of the alkylene group may be replaced by —O—, —S—, —NH—, or —CO—.), arylene (including heteroarylene), cycloalkylene, an alkoxyl group, a polyethylene glycol chain, and a group formed by arbitrarily bonding two or more groups selected from these groups.

[6] The compound or a salt thereof according to any one of [1] to [5], wherein the partial structure of an amino acid of L, together with C═O to which L is bonded, constitutes an amino acid, an amino acid residue, a peptide, or a part of an amino acid.

[7] The compound or a salt thereof according to any one of [1] to [6], wherein the partial structure of a saccharide of L′, together with O to which L′ is bonded, constitutes a saccharide, a part of a saccharide.

[8] The compound or a salt thereof according to any one of [1] to [7], wherein —Y in the general formula (I) is bonded to the ortho or para position of the benzene ring with respect to —C(R¹)(R²)X.

[9] The compound or a salt thereof according to any one of [1] to [8], wherein Y has a structure selected from the following.

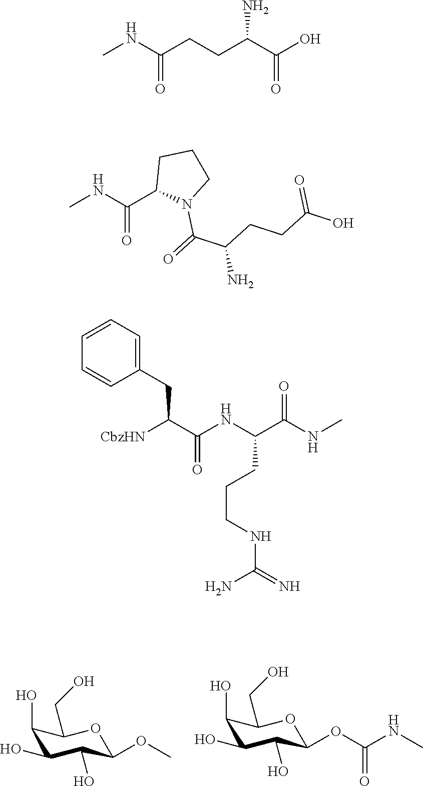

[10] The compound or a salt thereof according to any one of [1] to [9], wherein X is a fluorine atom or an ester group (—OCO—R′).

[11] The compound or a salt thereof according to any one of [1] to [10], wherein R¹ and R² are each independently selected from a hydrogen atom and a fluorine atom.

[12] The compound or a salt thereof according to any one of [1] to [11], wherein the monovalent substituent of R³ is selected from the group consisting of an alkyl group, an alkoxycarbonyl group (—CO—OR^{a}), a nitro group, an amino group, a hydroxyl group, an alkylamino group (—NHR^{a}, —NR^{a}₂), an alkoxy group (—OR^{a}), an ester group (—O—CO—R^{a}), a halogen atom, a boryl group, and a cyano group (R^{a} is a substituted or unsubstituted alkyl group or a substituted or unsubstituted aryl group, and when there are two or more R^{a}s, each R^{a} may be the same or different).

[13] The compound or a salt thereof according to [12], wherein the monovalent substituent of R³ is an alkyl group (for example, a methyl group) or an alkoxycarbonyl group (for example, a methoxycarbonyl group).

[14] The compound or a salt thereof according to [12], wherein the monovalent substituent of R³ is a halogen atom.

The compound or a salt thereof according to any one of [12] to [14], wherein at least one of the monovalent substituents of R³ is an alkyl group (for example, a methyl group) or an alkoxycarbonyl group (for example, a methoxycarbonyl group), and at least one of the monovalent substituents of R³ is a halogen atom.

[16] The compound or a salt thereof according to any one of [1] to [11], wherein both R³ and R⁴ are a hydrogen atom.

[17] A pharmaceutical composition comprising a compound according to any one of [1] to [16] or a pharmaceutically acceptable salt thereof.

[18] The pharmaceutical composition according to [17], which is used in nuclear medical examination.

[19] The pharmaceutical composition according to [18], that acts cell-selectively by cancer cell-specific enzyme activity to be accumulated in cancer cells.

[20] The pharmaceutical composition according to [19], wherein the enzyme is a peptidase or a glycosidase.

[21] The pharmaceutical composition according to any one of [18] to [20], wherein the nuclear medical examination is at least one selected from the group consisting of scintigraphy, SPECT (single-photon emission computed tomography), and PET (positron emission tomography).

[22] A method for diagnosing a disease or a condition that may lead to a disease, the method comprising:
(A) administering to a subject having or suspected of having a disease or condition a medicament comprising a compound according to any one of [1] to [15] or a pharmaceutically acceptable salt thereof; and
(B) examining the presence of one or more selected from the group consisting of a tumor, a cancer cell, and a cancer tissue in a target tissue or target organ of the subject by measuring radiation emitted from a radionuclide contained in the medicament localized in the target tissue or target organ.

[23] The diagnostic method according to [22], wherein the medicament is administered to the subject intravenously, intraperitoneally, or intratumorally.

[24] A kit including the compound of any one of [1] to [15] or a pharmaceutically acceptable salt thereof.

### Advantageous Effects of Invention

According to the present invention, it is possible to provide a novel compound that is promising as a probe for nuclear medical examination such as scintigraphy, SPECT (single-photon emission computed tomography), and PET (positron emission tomography).

### BRIEF DESCRIPTION OF DRAWINGS

FIG. 1 shows a schematic diagram showing that gGlu-4¹²⁵I-FMA, which is an example of a compound of the present invention, is retained inside a cell by a process of “metabolic trapping”.

FIG. 2 shows results of purification of gGlu-4¹²⁵I-FMA after ¹²⁵I labeling reaction performed using HPLC with a radiation detector.

FIG. 3 shows results of an enzymatic reaction between gGlu-4¹²⁵I-FMA and GGT performed using HPLC with a radiation detector. The upper left part shows a result before reaction with GGT, the upper right part shows a result after reaction with GGT, and the lower right part shows a result of detecting an unlabeled form of 2-amino-5-iodobenzylalcohol by PDA.

FIG. 4 shows a procedure of an intracellular uptake test of gGlu-4¹²⁵I-FMA.

FIG. 5 shows results of the intracellular uptake test of gGlu-4¹²⁵I-FMA probe.

FIG. 6 shows results of an intratumoral administration experiment of a probe to a subcutaneous tumor model mouse.

FIG. 7 shows a procedure of an experiment for visualizing peritoneal metastasis by intraperitoneal administration (i.p.) of a probe.

FIG. 8 shows results of the experiment for visualizing peritoneal metastasis by intraperitoneal administration (i.p.) of a probe. The left mouse is a peritoneal dissemination model mouse, and the right mouse is a cancer-free mouse. Both show a SPECT/CT image taken 5 hours after about 3 MBq of gGlu-4¹²⁵I-FMA was intraperitoneally administered.

FIG. 9 shows results of dissecting the peritoneal dissemination model mouse on the left side of FIG. 8, taking out intestines and mesentery, and imaging them by autoradiography (ARG).

### DESCRIPTION OF EMBODIMENTS

As used herein, the “halogen atom” means a fluorine atom, a chlorine atom, a bromine atom, or an iodine atom.

As used herein, the “alkyl” may be any aliphatic hydrocarbon group with a linear, branched, or cyclic structure, or a combination thereof. The number of carbon atoms in the alkyl group is not particularly limited, but is, for example, 1 to 6 (C1-6), 1 to 10 (C1-10), 1 to 15 (C1-15), or 1 to 20 (C1-20). When the number of carbon atoms is specified, it means an “alkyl” having a number of carbon atoms in that numerical range. For example, C1-8 alkyls include methyl, ethyl, n-propyl, isopropyl, n-butyl, isobutyl, sec-butyl, tert-butyl, n-pentyl, isopentyl, neo-pentyl, n-hexyl, isohexyl, n-heptyl, and n-octyl. As used herein, the alkyl group may have one or more arbitrary substituents. Examples of the substituent include, but are not limited to, alkoxy groups, halogen atoms, amino groups, mono- or di-substituted amino groups, substituted silyl groups, and acyl. When the alkyl group has two or more substituents, they may be the same or different. The same applies to alkyl moieties of other substituents including alkyl moieties (for example, alkoxy groups, arylalkyl groups, and the like).

As used herein, when it is defined that a certain functional group “may be substituted”, the type of substituent, the substitution position, and the number of substituents are not particularly limited, and when having two or more substituents, they may be the same or different. Examples of the substituent include, but are not limited to, alkyl groups, alkoxy groups, hydroxyl groups, carboxyl groups, halogen atoms, sulfo groups, amino groups, alkoxycarbonyl groups, and oxo groups. These substituents may further have substituents. Examples of such groups include, but are not limited to, halogenated alkyl groups and dialkylamino groups.

As used herein, the “aryl” may be either a monocyclic or condensed polycyclic aromatic hydrocarbon group, and may be an aromatic heterocyclic ring containing one or more heteroatoms (for example, an oxygen atom, a nitrogen atom, a sulfur atom, or the like) as ring-constituting atoms. In this case, the aryl may also be referred to as “heteroaryl” or “heteroaromatic”. The aryl may be attached at all possible positions, whether it is a monocyclic ring or a condensed ring. Non-limiting examples of the monocyclic aryl include a phenyl group (Ph), a thienyl group (2- or 3-thienyl group), a pyridyl group, a furyl group, a thiazolyl group, an oxazolyl group, a pyrazolyl group, a 2-pyrazinyl group, a pyrimidinyl group, a pyrrolyl group, an imidazolyl group, a pyridazinyl group, a 3-isothiazolyl group, a 3-isoxazolyl group, a 1,2,4-oxadiazol-5-yl group, and a 1,2,4-oxadiazol-3-yl group. Non-limiting examples of the condensed polycyclic aryl include a 1-naphthyl group, a 2-naphthyl group, a 1-indenyl group, a 2-indenyl group, a 2,3-dihydroinden-1-yl group, a 2,3-dihydroinden-2-yl group, a 2-anthryl group, an indazolyl group, a quinolyl group, an isoquinolyl group, a 1,2-dihydroisoquinolyl group, a 1,2,3,4-tetrahydroisoquinolyl group, an indolyl group, an isoindolyl group, a phthalazinyl group, a quinoxalinyl group, a benzofuranyl group, a 2,3-dihydrobenzofuran-1-yl group, a 2,3-dihydrobenzofuran-2-yl group, a 2,3-dihydrobenzothiophen-1-yl group, a 2,3-dihydrobenzothiophen-2-yl group, a benzothiazolyl group, a benzimidazolyl group, a fluorenyl group, and a thioxanthenyl group. As used herein, the aryl group may have one or more arbitrary substituents on the ring thereof. Examples of the substituent include, but are not limited to, alkoxy groups, halogen atoms, amino groups, mono- or di-substituted amino groups, substituted silyl groups, and acyl. When the aryl group has two or more substituents, they may be the same or different. The same applies to aryl moieties of other substituents including aryl moieties (for example, aryloxy groups, arylalkyl groups, and the like).

As used herein, the “alkoxy group” is a structure in which the alkyl group is bonded to an oxygen atom, and examples thereof include saturated linear, branched and cyclic alkoxy groups or combinations thereof. Examples of suitable groups include a methoxy group, an ethoxy group, a n-propoxy group, an isopropoxy group, a cyclopropoxy group, a n-butoxy group, an isobutoxy group, a s-butoxy group, a t-butoxy group, a cyclobutoxy group, a cyclopropylmethoxy group, a n-pentyloxy group, a cyclopentyloxy group, a cyclopropylethyloxy group, a cyclobutylmethyloxy group, a n-hexyloxy group, a cyclohexyloxy group, a cyclopropylpropyloxy group, a cyclobutylethyloxy group, and a cyclopentylmethyloxy group.

As used herein, the “alkylene” is a divalent group composed of a linear or branched saturated hydrocarbon, and examples thereof include methylene, 1-methylmethylene, 1,1-dimethylmethylene, ethylene, 1-methylethylene, 1-ethylethylene, 1,1-dimethylethylene, 1,2-dimethylethylene, 1,1-diethylethylene, 1,2-diethylethylene, 1-ethyl-2-methylethylene, trimethylene, 1-methyltrimethylene, 2-methyltrimethylene, 1,1-dimethyltrimethylene, 1,2-dimethyltrimethylene, 2,2-dimethyltrimethylene, 1-ethyltrimethylene, 2-ethyltrimethylene, 1,1-diethyltrimethylene, 1,2-diethyltrimethylene, 2,2-diethyltrimethylene, 2-ethyl-2-methyltrimethylene, tetramethylene, 1-methyltetramethylene, 2-methyltetramethylene, 1,1-dimethyltetramethylene, 1,2-dimethyltetramethylene, 2,2-dimethyltetramethylene, and 2,2-di-n-propyltrimethylene.

### 1. Compound Represented by General Formula (I) or Salt Thereof

One embodiment of the present invention is a compound represented by the following general formula (I) or a salt thereof (hereinafter, also referred to as the “compound of the present invention”).

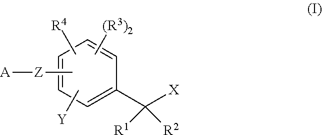

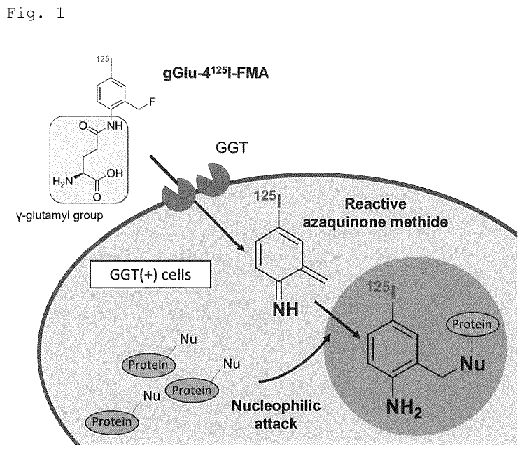

Without intending to be bound by theory, in the present invention, it has been found that a cancer biomarker enzyme is targeted, a substrate site thereof is incorporated into a drug molecule, an azaquinone methide reactive intermediate is produced only after the substrate site is cleaved by an enzymatic reaction, and the azaquinone methide reactive intermediate forms a covalent bond with a protein or the like in a cell, whereby the azaquinone methide reactive intermediate is metabolically trapped, and can be accumulated in cancer at a high concentration. FIG. 1 shows a schematic diagram showing that gGlu-4¹²⁵I-FMA, which is an example of the compound of the present invention, is retained inside a cell by a process of “metabolic trapping”.

In the general formula (I), Y is an enzyme recognition site, a part of which is cleaved by cancer cell-specific enzyme activity to induce formation of quinone methide.

Y can be selected according to the type of the target enzyme. When the cancer biomarker enzyme as the target enzyme is a peptidase, Y is selected from groups derived from amino acids and groups including amino acids, and when the target enzyme is a glycosidase, Y is selected from groups derived from saccharides.

In the general formula (I), Y is preferably —NH—CO-L, —NH-L′, or —OL′.

Here, L is a partial structure of an amino acid. The partial structure of an amino acid of L means that L, together with C═O to which L is bonded, constitutes an amino acid, an amino acid residue, a peptide, or a part of an amino acid.

As used herein, as for the “amino acid”, any compound can be used as long as it is a compound having both an amino group and a carboxyl group, and the amino acid includes natural and non-natural compounds. The amino acid may be any of a neutral amino acid, a basic amino acid, and an acidic amino acid, and it is possible to use amino acids which themselves function as a transmitter such as a neurotransmitter, and amino acids which are constituents of polypeptide compounds such as physiologically active peptides (including dipeptides, tripeptides, tetrapeptides, and oligopeptides) or proteins. The amino acid may be, for example, an a amino acid, a β amino acid, a γ amino acid, or the like. It is preferable to use an optically active amino acid as the amino acid. For example, as the α amino acid, either a D- or L-amino acid may be used, but it may be preferable to select an optically active amino acid that functions in a living body.

As used herein, the “amino acid residue” refers to a structure corresponding to the remaining partial structure obtained by removing a hydroxyl group from a carboxyl group of an amino acid.

Amino acid residues include residues of a amino acids, residues of β amino acids, and residues of γ amino acids. Examples of preferred amino acid residues include a γ-glutamyl group of a GGT substrate and a dipeptide (dipeptide composed of an amino acid-proline)) of a DPP4 substrate.

As used herein, the “peptide” refers to a structure in which two or more amino acids are linked by a peptide bond.

Examples of preferred peptides include the dipeptide of a DPP4 substrate (dipeptide composed of an amino acid-proline; here, the amino acid is, for example, glycine, glutamic acid, or proline) described above.

Examples of the case in which L constitutes a part of an amino acid together with C═O to which L is bonded include a structure in which a carboxyl group of the side chain of the amino acid is bonded to —NH₂ to form a carbonyl group to be a part of the amino acid as in the γ-glutamyl group described above.

L′ is a saccharide or a partial structure of a saccharide, a saccharide or a partial structure of a saccharide having a self-cleaving linker, or an amino acid or a peptide having a self-cleaving linker.

Here, the partial structure of a saccharide of L′ refers to a structure corresponding to the remaining partial structure obtained by removing one hydroxyl group from the saccharide. The partial structure of a saccharide, together with O to which L′ is bonded, constitutes a saccharide, a part of a saccharide.

Examples of the saccharide include β-D-glucose, β-D-galactose, β-L-galactose, β-D-xylose, α-D-mannose, β-D-fucose, α-L-fucose, β-L-fucose, β-D-arabinose, β-L-arabinose, β-D-N-acetylglucosamine, and β-D-N-acetylgalactosamine, and β-D-galactose is preferred.

The self-cleaving linker means a linker that is spontaneously cleaved and decomposed, and examples thereof include carbamates, urea, a para-aminobenzyloxy group, and ester groups (—CO—O—, —O—CO—).

In one preferred aspect of the present invention, Y has a structure selected from the following.

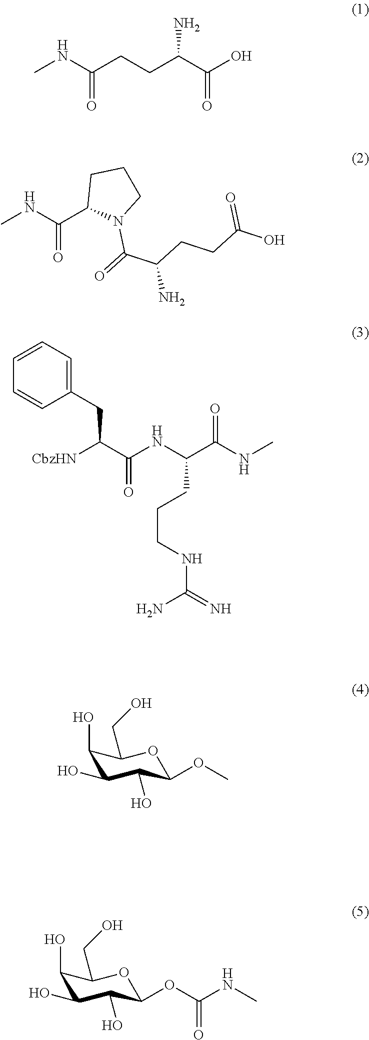

In the general formula (I), X acts as a leaving group that leaves from a benzene ring when a part of the enzyme recognition site of Y is cleaved by cancer cell-specific enzyme activity, resulting in formation of quinone methide.

X is selected from the group consisting of a fluorine atom, an ester group (—OC(═O)—R′), a carbonate group (—OCO₂—R′), a carbamate group (—OCONH—R′), phosphoric acid and its ester group (—OP(═O)(—OR′)(—OR″), and sulfuric acid and its ester group (—OSO₂—OR′).

Here, R′ and R″ are each independently selected from substituted or unsubstituted alkyl groups and substituted or unsubstituted aryl groups.

X is preferably a fluorine atom or an ester group (—OCO—R′). Without intending to be bound by theory, when X is a fluorine atom or an ester group (—OC(═O)—R′), the quinone methide is quickly formed upon cleavage of Y.

R¹ and R² are each independently selected from a hydrogen atom and a monovalent substituent. The monovalent substituent is a halogen atom or an alkyl group having 1 or more carbon atoms (for example, an alkyl group having about 1 to 6 carbon atoms).

R¹ and R² are preferably each independently selected from a hydrogen atom and a fluorine atom.

—Y in the general formula (I) is preferably bonded to the ortho or para position of the benzene ring with respect to —C(R¹)(R²) X. When —Y and —C(R¹)(R²)X have such a positional relationship on the benzene ring, a quinone methide structure can be formed when Y is cleaved.

R³ is a hydrogen atom or one to two identical or different monovalent substituents present on the benzene ring.

The monovalent substituent of R³ is selected from the group consisting of an alkyl group having 1 or more carbon atoms (for example, an alkyl group having about 1 to 6 carbon atoms), an alkoxycarbonyl group (—C(═O)—OR^{a}), a nitro group, an amino group, a hydroxyl group, an alkylamino group (—NHR^{a}, —NR^{a}₂), an alkoxy group (—OR^{a}), an ester group (—O—CO—R^{a}), an amide group (—NHCOR^{a}), a halogen atom, a boryl group, and a cyano group. Here, R^{a} is a substituted or unsubstituted alkyl group or a substituted or unsubstituted aryl group. When there are two or more R^{a}, each R^{a} may be the same or different.

In one aspect of the compound of the present invention, the monovalent substituent of R³ is an alkyl group (for example, a methyl group) or an alkoxycarbonyl group (for example, a methoxycarbonyl group). It is preferable to introduce an alkyl group which is an electron-donating group into the benzene ring because the alkyl group is expected to promote intracellular retention.

In one aspect of the compound of the present invention, the monovalent substituent of R³ is a halogen atom (preferably, an iodine atom). When R³ is a halogen atom (preferably, an iodine atom), it is possible to enhance the trapping effect on cells.

In one aspect of the compound of the present invention, at least one of the monovalent substituents of R³ is an alkyl group (for example, a methyl group) or an alkoxycarbonyl group (for example, a methoxycarbonyl group), and at least one of the monovalent substituents of R³ is a halogen atom.

When R³ is the monovalent substituent described above, particularly an alkyl group, the position of R³ is preferably the 5-position corresponding to the para position and/or the 4-position corresponding to the meta position of —C(R¹)(R²)X.

In another aspect of the compound of the present invention, all of R³ are hydrogen atoms.

R⁴ in the general formula (I) is a hydrogen atom, or a substituent or molecule capable of altering pharmacokinetics.

The substituent or molecule capable of altering pharmacokinetics may be any substituent or molecule known to alter pharmacokinetics. Examples of such a substituent or molecule include structures that are known to bind to serum albumin, such as substituted or unsubstituted biphenyl groups; monovalent or divalent substituents derived from a bicyclic compound (for example, naphthalene, quinoline, or the like); dye molecules such as Evans blue; and monovalent or divalent substituents derived from p-iodophenylbutyric acid.

Here, the monovalent substituent derived from a bicyclic compound means a monovalent substituent obtained by removing one hydrogen from the bicyclic compound (for example, a naphthyl group), and the divalent substituent derived from a bicyclic compound means a divalent substituent obtained by removing two hydrogens from the bicyclic compound. The monovalent or divalent substituent derived from a bicyclic compound may be unsubstituted or may have a substituent. Examples of these substituents include alkyl groups, alkoxy groups, hydroxyl groups, carboxyl groups, halogen atoms, sulfo groups, amino groups, alkoxycarbonyl groups, and oxo groups.

In addition, the substituent or molecule capable of altering pharmacokinetics also includes groups formed by bonding the same or different two or more substituents or molecules mentioned above optionally via a linking group. Also included in the substituent or molecule capable of altering pharmacokinetics are, for example, groups formed by bonding two or more identical or different substituted or unsubstituted biphenyl groups optionally via a linking group; groups formed by bonding two or more identical or different substituted or unsubstituted naphthyl groups optionally via a linking group; and groups formed by bonding one or more substituted or unsubstituted biphenyl groups and one or more substituted or unsubstituted naphthyl groups (when there are two or more of either or both of them, they may be the same or different) optionally via a linking group (however, the present invention is not limited thereto).

The linking group may be any group that has a function as a linker and is metabolically stable, and is preferably selected from the group consisting of an alkylene group (with the proviso that one or more —CH₂— of the alkylene group may be replaced by —O—, —S—, —NH—, or —CO—.), arylene (including heteroarylene), cycloalkylene (for example, cyclohexylene), an alkoxyl group, a polyethylene glycol chain, and a group formed by arbitrarily bonding two or more groups selected from these groups.

By introducing such a substituent or molecule capable of altering pharmacokinetics into the benzene ring, it becomes easy to increase and/or adjust the half-life in blood, and the degree of freedom of the administration route can be increased when the pharmaceutical composition or the like containing the compound of the present invention is administered to a subject.

The substituent or molecule capable of altering pharmacokinetics described above can be introduced into the benzene ring via a linker or directly.

The linker is selected from the group consisting of an alkylene group (with the proviso that one or more —CH₂— of the alkylene group may be replaced by —O—, —S—, —NH—, or —CO—.), arylene (including heteroarylene), cycloalkylene, an alkoxyl group, a polyethylene glycol chain, and a group formed by arbitrarily bonding two or more groups selected from these groups.

In one aspect of the compound of the present invention, R⁴ is a hydrogen atom.

In one aspect of the compound of the present invention, both R³ and R⁴ are a hydrogen atom.

In the general formula (I), A represents a radionuclide. The radionuclide is selected from the group consisting of ¹²⁵I, ²¹¹At, ¹⁸F, ¹⁵O, ¹²³I, ¹³¹I, ¹²⁴I, and ¹¹C.

In the general formula (I), Z represents a single bond or a linking group.

Here, the case where Z is a “single bond” means that A is directly bonded to the benzene ring without interposing a linking group.

The linking group may be any group that has a function as a linker and is metabolically stable, and is preferably selected from the group consisting of an alkylene group (with the proviso that one or more —CH₂— of the alkylene group may be replaced by —O—, —S—, —NH—, or —CO—.), arylene (including heteroarylene), cycloalkylene (for example, cyclohexylene), an alkoxyl group, a polyethylene glycol chain, and a group formed by arbitrarily bonding two or more groups selected from these groups.

The number of carbon atoms in the alkylene group is not particularly limited, but is preferably 5 to 20, and more preferably 5 to 15. Even when —CH₂— in the alkylene group is replaced by —O—, —S—, —NH—, or —CO—, these groups are considered to have one carbon and are included in “the number of carbon atoms in the alkylene group” described above.

Also, arylene includes those having a benzene ring such as a phenylene group as a linker, and divalent linkers derived from an aromatic or cyclic hydrocarbon containing a heterocyclic ring.

In one preferred aspect of the compound of the present invention, the linking group is an alkylene group (with the proviso that one or more —CH₂— of the alkylene group may be replaced by —O—, —S—, —NH—, or —CO—.).

The position at which A-Z— is introduced is not particularly limited, but it is preferable that A-Z— is bonded to the meta or para position of the benzene ring with respect to Y because this position is metabolically stable and there is a possibility that the compound does not serve as a substrate of the target enzyme when A-Z— is too close to the enzyme recognition site.

Non-limiting examples of the compound of the present invention are shown below, but the compound of the present invention is not limited thereto.

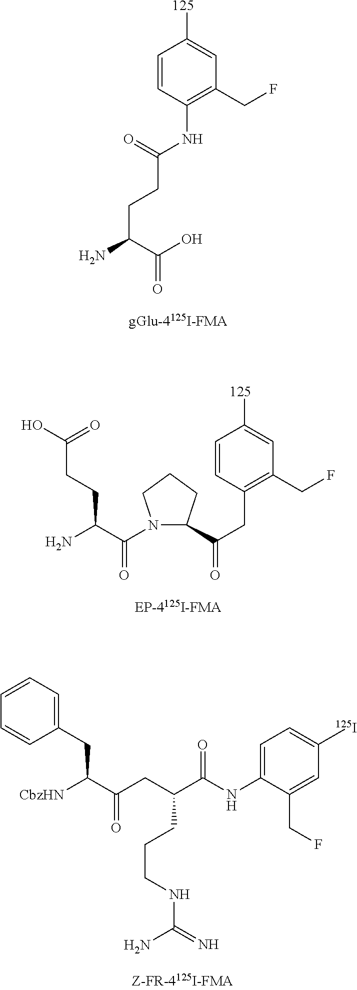

Unless otherwise noted, the compound represented by the general formula (I) also includes stereoisomers such as tautomers, geometric isomers (for example, E-isomer, Z-isomer, and the like), and enantiomers thereof. That is, when one or more asymmetric carbons are contained in the compound represented by the general formula (I), the asymmetric carbons can each independently have either an (R) configuration or an(S) configuration as to the stereochemistry, and the compound may exist as a stereoisomer such as an enantiomer or a diastereoisomer of the derivative. Therefore, any stereoisomer in pure form, any mixture of stereoisomers, racemates, and the like can be used as an active ingredient of a probe for nuclear medical examination of the present invention, and all are encompassed in the scope of the present invention.

The method for producing the compound represented by the general formula (I) is not particularly limited, but synthesis methods for representative compounds among the compounds encompassed by the general formula (I) are specifically shown in the examples in the present specification. A person skilled in the art can produce compounds encompassed by the formula (I) by appropriately changing or modifying starting raw materials, reaction reagents, reaction conditions, and the like as necessary while referring to the examples in the present specification and the following scheme.

### 2. Pharmaceutical Composition, Nuclear Medical Diagnostic Imaging Agent, and Imaging Reagent

The compound of the present invention contains a radionuclide, and thus can be used for pharmaceutical compositions, nuclear medical image diagnosis, and imaging.

Therefore, another embodiment of the present invention is a pharmaceutical composition containing a compound of the present invention or a pharmaceutically acceptable salt thereof (hereinafter also referred to as the “pharmaceutical composition of the present invention”).

A preferred embodiment of the pharmaceutical composition of the present invention is a pharmaceutical composition used in nuclear medical examination.

Examples of the nuclear medical examination include scintigraphy, SPECT (single-photon emission computed tomography), and PET (positron emission tomography).

Another embodiment of the present invention is a nuclear medical diagnostic imaging agent containing a compound of the present invention or a pharmaceutically acceptable salt thereof.

As used herein, the “nuclear medical diagnostic imaging agent” refers to a medicament (drug) containing the compound of the present invention used in in vivo nuclear medical examination in which the medicament is administered into a body, and radiation (radioactive signal) emitted from the body is measured and imaged from outside the body to evaluate or examine a biological function of an organ or a tissue, diagnose a disease, or the like, or a medicament containing the compound of the present invention used in in vitro nuclear medical examination in which the medicament is reacted in a test tube with a sample of a tissue, blood, or the like collected from the body to evaluate or examine a biological function of an organ or a tissue, diagnose a disease, or the like. Examples of the in vivo nuclear medical examination include methods using a nuclear medical imaging probe such as scintigraphy, SPECT (single-photon emission computed tomography), and PET (positron emission tomography described above.

Also, another embodiment of the present invention is an imaging reagent containing a compound of the present invention or a pharmaceutically acceptable salt thereof.

As used herein, the “imaging” includes administering into a body a compound of the present invention containing a radioisotope (RI), that is, a radionuclide (imaging probe), and measuring and imaging radiation (radioactive signal) emitted from the body from outside the body. Also, in another embodiment, the “imaging” includes measuring and imaging from outside a body radiation (radioactive signal) emitted from the living body to which the compound of the present invention containing a radioisotope (RI) (imaging probe) is administered. The “imaging” includes obtaining measurement data and/or image data for diagnostic nuclear medical imaging.

The pharmaceutical composition of the present invention, the nuclear medical diagnostic imaging agent, and the imaging reagent (hereinafter, these are also collectively referred to as “the pharmaceutical composition of the present invention and the like”) preferably acts cell-selectively by cancer cell-specific enzyme activity to be accumulated in cancer cells.

In the pharmaceutical composition of the present invention and the like, the cancer cell-specific enzyme is preferably a peptidase or a glycosidase.

Examples of the peptidase include γ-glutamyl transpeptidase (GGT), dipeptidyl peptidase IV (DPP-IV), cathepsin B/L, and calpain.

Examples of the glycosidase include β-galactosidase, β-glucosidase, α-mannosidase, α-L-fucosidase, β-hexosaminidase, and β-N-acetylgalactosaminidase.

The pharmaceutical composition of the present invention and the like may contain not only the compound represented by the general formula (I) but also a salt thereof or a solvate or hydrate of the compound or the salt. The salt is not particularly limited as long as it is a pharmaceutically acceptable salt, and examples thereof include base addition salts, acid addition salts, and amino acid salts. Examples of the base addition salt include alkaline earth metal salts such as sodium salts, potassium salts, calcium salts, and magnesium salts, ammonium salts, or organic amine salts such as triethylamine salts, piperidine salts, and morpholine salts, and examples of the acid addition salt include mineral acid salts such as hydrochlorides, hydrobromides, sulfates, nitrates, and phosphates; and organic acid salts such as methanesulfonic acid, benzenesulfonic acid, p-toluenesulfonic acid, acetic acid, propionates, tartaric acid, fumaric acid, maleic acid, malic acid, oxalic acid, succinic acid, citric acid, benzoic acid, mandelic acid, cinnamic acid, lactic acid, glycolic acid, glucuronic acid, ascorbic acid, nicotinic acid, and salicylic acid. Examples of the amino acid salt include glycine salts, aspartic acid salts, and glutamic acid salts. In addition, metal salts such as aluminum salts are also acceptable.

The type of solvent for forming a solvate is not particularly limited, and examples thereof include solvents such as ethanol, acetone, and isopropanol.

The pharmaceutical composition of the present invention is used for nuclear medical examination (preferably, at least one selected from the group consisting of scintigraphy, SPECT (single-photon emission computed tomography), and PET (positron emission tomography)).

That is, the pharmaceutical composition of the present invention is administered into a body of a human or an animal other than a human (mouse, rat, hamster, rabbit, cat, dog, cow, sheep, monkey, or the like), and is used for evaluating or examining the biological function of an organ or tissue by measuring and imaging radiation (radioactive signal) emitted from the body from outside the body.

Examples of the disease to be evaluated or examined include, but are not limited to, brain tumor, malignant melanoma, head and neck cancer, breast cancer, liver cancer, gastric cancer, colon cancer, pancreatic cancer, ovarian cancer, lung cancer, kidney cancer, prostate cancer, testicular cancer, glioblastoma, sarcoma, bone cancer, brain cancer, head and neck cancer, skin cancer, thyroid cancer, bladder cancer, mesothelioma, meningioma, and sarcoma.

When used as the pharmaceutical composition and the like containing the compound represented by the general formula (I) or a pharmaceutically acceptable salt thereof (that is, the pharmaceutical composition, the nuclear medical diagnostic imaging agent, and the imaging reagent), they can be formulated by mixing them with a pharmaceutically acceptable carrier or diluent according to a known method. The dosage form is not particularly limited, and may be a pharmaceutical composition for oral administration in the form of an injection, a tablet, a powder, a granule, a capsule, a solution, a suppository, a sustained-release agent, or the like.

Administration of the pharmaceutical composition of the present invention and the like may be local or systemic. The administration route can be appropriately determined according to the condition of the subject and the like, and for example, the composition can also be prepared as a pharmaceutical composition for parenteral administration in the form of an injection for intravenous administration, intraarterial administration, intradermal administration, intramuscular administration, intraperitoneal administration, intratumoral administration, or the like.

The dosage (dose) of the pharmaceutical composition of the present invention and the like is not particularly limited, and may be a sufficient amount for obtaining a desired contrast for imaging, and can be, for example, 1 μg or less.

The pharmaceutical composition of the present invention and the like may be a formulation in the form of a solution or a powder. These formulations are prepared according to a conventional method. The liquid formulation may be dissolved or suspended in water or another appropriate solvent at the time of use. Also, the tablet and granule may be coated by a well-known method. In the case of an injection, it is prepared by dissolving the compound of the present invention in water, but may be dissolved in physiological saline or a glucose solution as necessary, or a buffer or a preservative may be added.

### 3. Diagnostic Methods

Another aspect of the present invention is a method for diagnosing a disease or a condition that may lead to a disease, the method including:
(A) administering to a subject having or suspected of having a disease or condition a medicament containing a compound of the present invention or a pharmaceutically acceptable salt thereof; and
(B) examining the presence of one or more selected from the group consisting of a tumor, a cancer cell, and a cancer tissue in a target tissue or target organ of the subject by measuring radiation emitted from a radionuclide contained in the medicament localized in the target tissue or the target organ
(hereinafter, also referred to as the “diagnostic method of the present invention”).

Here, the medicament refers to any one of the pharmaceutical composition of the present invention, the nuclear medical diagnostic imaging agent, and the imaging reagent described above.

The subject is not particularly limited, but is a human or an animal other than a human (mouse, rat, hamster, rabbit, cat, dog, cow, sheep, monkey, or the like).

The subject having or suspected of having a disease or condition includes a subject having or suspected of having cancer.

Administration of the medicament of the present invention may be oral administration or parenteral administration. Also, parenteral administration may be local or systemic. The administration route can be appropriately determined according to the condition of the subject or the like, and can be performed, for example, by administering an injection for intravenous administration, intraarterial administration, intradermal administration, intramuscular administration, intraperitoneal administration, intratumoral administration, or the like.

As used herein, the term “cancer” or “tumor” refers to any neoplastic proliferation in a subject, including early tumors and any metastases. The cancer can be a liquid or solid tumor type. Liquid tumors include tumors of hematologic origin, including, for example, myeloma (for example, multiple myeloma), leukemia (for example, Waldenstrom's syndrome, chronic lymphocytic leukemia, other leukemias), and lymphoma (for example, B cell lymphoma, non-Hodgkin's lymphoma). Solid tumors can occur in organs and include cancers of the lung, brain, breast, prostate, ovary, colon, kidney, and liver.

Types of cancer cells or cancer tissues to be subjected to the diagnostic method of the present invention include cells or tissues of brain tumor, malignant melanoma, head and neck cancer, breast cancer, liver cancer, gastric cancer, colon cancer, pancreatic cancer, ovarian cancer, lung cancer, kidney cancer, prostate cancer, testicular cancer, glioblastoma, sarcoma, bone cancer, brain cancer, head and neck cancer, skin cancer, thyroid cancer, bladder cancer, mesothelioma, meningioma, and sarcoma.

As used herein, the term “cancer tissue” means any tissue including cancer cells.

The term “tissue” should be construed in the broadest sense, including part or all of an organ, and should not be construed in any limiting sense.

As one aspect of the diagnostic method of the present invention, the method includes detecting a radioactive signal of a compound of the present invention from a subject to which the medicament containing the compound or a pharmaceutically acceptable salt thereof is previously administered. The signal detection is preferably performed, for example, after a sufficient time for signal detection has elapsed after the compound is administered.

In one aspect of the diagnostic method of the present invention, the method includes reconstructing and converting the detected signal into an image and displaying the image, and/or quantifying the detected signal for presenting an accumulation amount. As used herein, the “displaying” includes displaying on a monitor and/or printing. As used herein, the “presenting” includes storing the calculated accumulation amount and/or outputting the accumulation amount to the outside.

The signal detection can be appropriately determined according to the type of radionuclide of the compound of the present invention to be used, and can be performed using, for example, scintigraphy, SPECT, PET, or the like. Scintigraphy and SPECT include, for example, measuring γ-rays emitted from a subject to which a radioactive compound according to the present disclosure has been administered by a gamma camera. The measurement by the gamma camera includes, for example, measuring radiation (γ-rays) emitted from the radionuclide of the administered compound in a certain time unit, and preferably includes measuring a direction in which the radiation is emitted and a radiation quantity in a certain time unit. The diagnostic method of the present invention may further include representing the distribution of the measured radioactive compound obtained by the measurement of radiation as a cross-sectional image and reconstructing the obtained cross-sectional image.

For example, PET may include simultaneously counting gamma rays generated from a subject to which a radioactive compound according to the present disclosure has been administered by pair annihilation of a positron and an electron with a detector for PET, and may further include depicting a three-dimensional distribution of positions of a radionuclide emitting a positron based on the measurement result.

X-ray CT and/or MRI measurement may be performed in conjunction with scintigraphy, SPECT, or PET measurement. Thereby, for example, a fused image in which an image obtained by scintigraphy, SPECT, or PET (functional image) and an image obtained by CT or MRI (morphological image) are fused can be obtained.

Another embodiment of the present invention is a kit including a compound of the present invention or a pharmaceutically acceptable salt thereof (hereinafter also referred to as the “kit of the present invention”). The kit of the present invention is used in nuclear medical examination.

The kit of the present invention can further include one or more selected from components for preparing the probe of the present invention such as a buffer and an osmotic pressure regulator, and an instrument used for administration of a compound such as a syringe.

### EXAMPLES

The present invention is described in more detail below using examples, but the scope of the present invention is not limited to these examples.

### Materials

Reagents (seller, product code)
Bis(tributyltin) (Sigma-Aldrich, 251127-10G)
Tris(dibenzylideneacetone)dipalladium(0) (Wako, 209-18401)
N,N-Dimethylformamide, deoxidized (DMF) (Wako, 042-32071)
Acetic acid (AcOH) (wako, 017-00256)
Methanol (MeOH) (wako, 131-01826)
N-Chlorosuccinimide (NCS) (wako, 354-13862)
Iodine-125 radionuclide (Perkin Elmer, Inc., NEZ033A)
Acetonitrile (Sigma-Aldrich, 34888-2.5L)
Formic acid (FA) (nacalai tesque, 08965-82)
GGT(γ-GT) (wako, 307-50673)

### Measuring Instrument

Automated Flash Chromatography System (Yamazen, AI-580)

Preparative HPLC (JASCO, LC-2000Plus series)
Preparative C18 column (GL Sciences, 5020-06822)
Shaker (eppendorf Thermomixer coomfort)
Analytical HPLC (SHIMADZU, LC-20AB)
Analytical C18 column (GL Sciences, 5020-07345)
Radioactivity-HPLC-flow monitor (GABI Star, Elysia-raytest)
Curiemeter (ALOKA CURIEMETER IGC-8, HITACHI)
γ Counter (2480 Wizard2, Perkin Elmer)
SPECT/CT (NanoSPECT/CT, Bioscan)

### Synthesis Example 1

According to the following Scheme 1, gGlu-4¹²⁵I-FMA, which is a compound of the present invention, was synthesized.

### Scheme 1

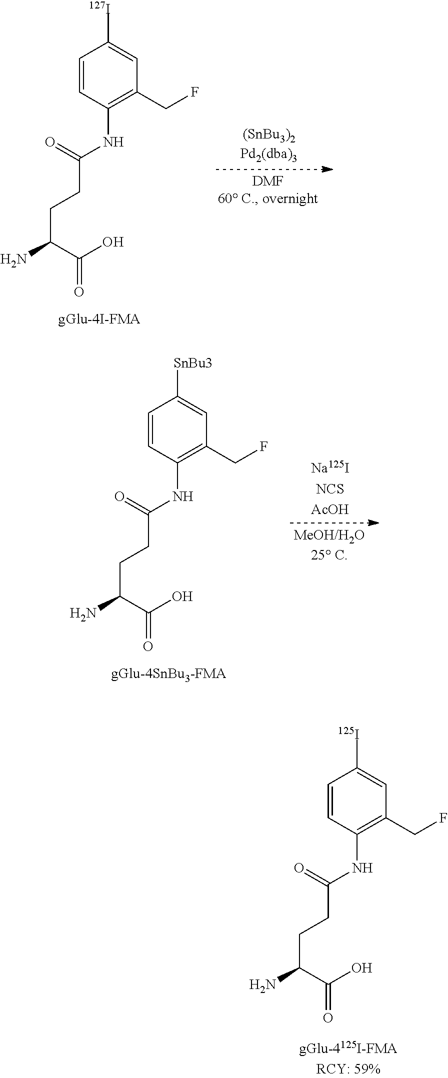

<Synthesis of gGlu-4SnBu₃-FMA>

gGlu-4I-FMA (150.4 mg, 0.396 mmol) was charged into a 50 mL two-necked recovery flask, and the flask was purged with argon. gGlu-4I-FMA was dissolved in 5 mL of deoxygenated DMF, bis(tributyltin) (1.5 mL, 1.148 g/mL, 2.97 mmol, 7.5 eq) was added thereto, and degassing and argon substitution were repeated three times. Tris(dibenzylideneacetone)dipalladium (0) (36.3 mg, 0.0396 mmol, 0.1 eq) was added thereto, degassing and argon substitution were performed again three times, and then the mixture was reacted at 60° C. overnight (20 hours). After the reaction, the reaction mixture was allowed to cool to room temperature, filtered through Celite with MeOH, and the filtrate was concentrated with an evaporator.

The obtained crude was subjected to flash column chromatography under the condition that hexane/ethyl acetate=50/50 was allowed to flow for 10 minutes followed by MeOH for 10 minutes to remove lipid-soluble impurities, and the resultant was concentrated by an evaporator and then purified by preparative HPLC. With H₂O as liquid A and MeCN+1% H₂O as liquid B, purification was performed with a gradient of A/B=40/60 to 0/100 (40 min) at a flow rate of 10 mL/min to obtain 11.1 mg (yield 5%) of gGlu-4SnBu₃-FMA.

<Synthesis of gGlu-4¹²⁵I-FMA (¹²⁵I labeling)>

A Na¹²⁵I solution (8.90 MBq in 6.5 μL 10⁻⁵ M NaOH aq.) was dispensed into a 1.5 mL Eppendorf tube. To this solution, 0.5 μL of a solution obtained by dissolving about 2 mg of NCS in 1000 μL of MeOH, 10 μL of a solution obtained by dissolving 2.7 mg of gGlu-4SnBu₃-FMA in 540 μL of MeOH, and 1.7 μL of AcOH were added in this order, and the mixture was stirred at 25° C. and 500 rpm for 30 minutes using a shaker. The entire amount of this solution was injected into HPLC with a Radioactivity-HPLC-flow monitor (GABI Star) to perform purification. Details regarding purification will be described below. Also when labeling is performed using Na¹²⁵I having higher radioactivity such as 100 MBq, the labeling reaction can be performed according to the same procedure.

<Device data of gGlu-4SnBu₃-FMA>

1H NMR (400 MHZ, CD3OD): δ 0.80 (t, 9H, J=7.3 Hz), 1.00 (t, 6H, J=8.2 Hz), 1.19-1.30 (m, 6H), 1.43-1.51 (m, 6H), 2.10 (dt, 2H, J=7.0, 6.6 Hz), 2.59 (t, 2H, 7.4 Hz), 3.65 (t, 1H, J=6.2 Hz), 5.29 (d, 2H, J=48 Hz), 7.28 (d, 1H, J=7.7 Hz), 7.37 (d, 1H, J=7.8 Hz), 7.43 (s, 1H)

HRMS (ESI+): Calculated for [M+Na]+, 567.20197, Found, 567.20011 (1.9 mDa)

<Device data of gGlu-4I-FMA (preparation of cold, TFA salt)>

1H NMR (400 MHZ, CD₃OD): δ 2.20 (m, 2H), 2.74 (t, 2H, J=7.1 Hz), 4.09 (t, 1H, 6.5 Hz), 5.36 (d, 2H, J=47 Hz), 7.23 (d, 1H, J=8.4 Hz), 7.72 (d, 1H, J=8.4 Hz), 7.82 (s, 1H)

13C NMR (100 MHZ, CD₃OD): 26.9, 32.5, 53.4, 81.6 (d, J=165 Hz), 91.2, 128.8, 135.0 (d, J=17 Hz), 135.9 (d, J=3.9 Hz), 138.2 (d, J=8.8 Hz), 139.3 (d, J=2.2 Hz), 171.5, 173.2

HRMS (ESI+): Calculated for [M+H]+, 381.01059, Found, 381.01103 (−0.4 mDa); Calculated for [M+Na]+, 402.99253, Found, 402.99272 (−0.2 mDa)

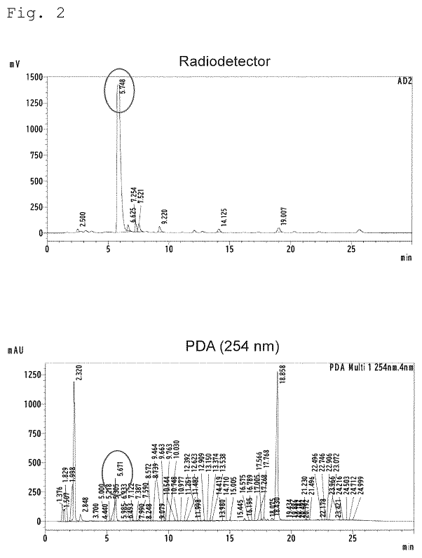

Purification of gGlu-4¹²⁵I-FMA was performed using HPLC with a radiation detector. The results are shown in FIG. 2. For purification, the analytical HPLC described above was used. With H₂O+0.1% FA as liquid A and MeCN/H₂O=8/2+0.1% FA as liquid B, purification was performed with a gradient of A/B=70/30 to 0/100 (20 min) at a flow rate of 1 mL/min. The upper figure is a result of monitoring with a radiation detector, and the lower figure is a result of monitoring with PDA at 254 nm. Note that there is a lag of about 10 seconds due to the gap of the flow path between PDA and radiation detector. A peak at a retention time of 5.7 minutes circled in the figure was collected in a pear-shaped flask. The purified gGlu-4¹²⁵I-FMA was dried overnight in a freeze dryer.

### Example 1

HPLC Analysis of gGlu-4¹²⁵I-FMA and metabolites thereof

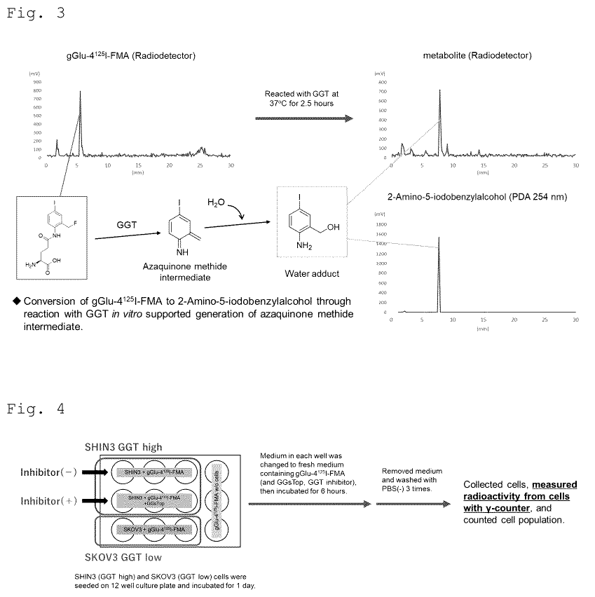

The purified gGlu-4¹²⁵I-FMA was dissolved in DPBS(-) to prepare about 5 kBq/μL. The result of the radiation detector when 20 μL of DPBS(-) was added to 5 μL of this solution and the entire amount of 25 μL was injected into the analytical HPLC is shown in the upper left of FIG. 3. Here, the analysis conditions of HPLC are exactly the same as the conditions at the time of purification in FIG. 2.

For analysis of metabolites, 40 μL of a 100 U/mL GGT solution (final concentration: 80 U/mL) was added to 10 μL of the above gGlu-4¹²⁵I-FMA solution, and the mixture was stirred at 37° C. and 500 rpm for 3.5 hours using a shaker. The result of the radiation detector when a half amount 25 μL from this reaction liquid was injected for analysis is shown in the upper left of FIG. 3. The lower right is a result of extracting 254 nm with PDA when a cold preparation of 2-amino-5-iodobenzylalcohol obtained by producing azaquinone methide after gGlu-4¹²⁵I-FMA was metabolized by GGT, and reacting azaquinone methide with water that would be the main nucleophile in this enzymatic reaction experiment was analyzed under the same analysis conditions.

The reaction with GGT has shifted the retention time from 5.4 min to 7.5 min, and the product after the reaction has the same retention time as the cold preparation of 2-amino-5-iodobenzylalcohol, suggesting that gGlu-4¹²⁵I-FMA reacts with GGT to be converted to 2-amino-5-iodobenzylalcohol, and this result supports production of an azaquinone methide intermediate by a metabolic enzyme reaction.

### Example 2

Evaluation of intracellular uptake of gGlu-4¹²⁵I-FMA probe using cells with high/low GGT activity and GGT inhibitor

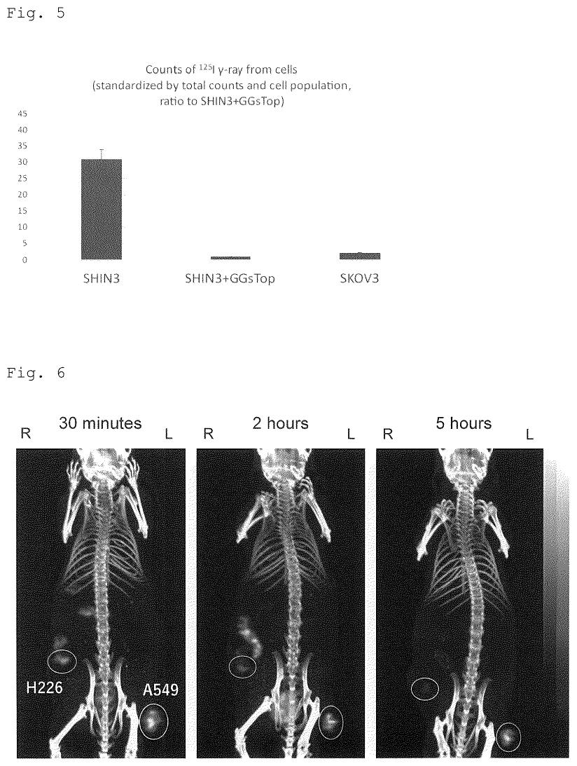

Intracellular uptake of gGlu-4¹²⁵I-FMA was evaluated using cells with high or low GGT activity and a GGT inhibitor, GGsTop, by the procedure shown in FIG. 4.

First, SHIN3 (high GGT activity) and SKOV3 (low GGT activity) cells were seeded in 12 well culture plates and incubated for 1 day. Next, the medium of each well was changed to fresh medium containing gGlu-4¹²⁵I-FMA (and GGsTop), and the cells were incubated for 6 hours. Thereafter, the medium was removed and the plates were washed three times with PBS(-). Then, the cells were collected, radioactivity from the cells was measured with a Y-counter, and the number of cells was counted. The results are shown in FIG. 5.

FIG. 5 shows counts of ¹²⁵I γ-rays from cells as measured with a gamma counter (ratio to SHIN3+GGsTop, standardized by the number of cells and total counts of all fractions combining media and three washes). Since there was almost no radioactivity in the third wash fraction and the radioactivity from the cell fraction was much higher than that, it is considered that the radioactivity from the cell fraction is derived from the nuclide retained in the cell.

In SHIN3, the uptake rate in the case of not adding an inhibitor is about 30 times as compared with the case of adding an inhibitor, and in SKOV3 with low GGT activity, the uptake rate is suppressed to be low, and thus it is found that the present probe is taken up into cells in a GGT activity-dependent manner. In addition, since a signal remained in the cell even when the wash operation was performed, it is considered that the incorporated probe remains in the cell at a certain ratio and withstands washout.

### Example 3

Intratumoral administration experiment of probe to subcutaneous tumor model mouse

On the basis of the results of Example 2, a probe was intratumorally administered to a subcutaneous tumor model mouse, and SPECT/CT imaging was performed.

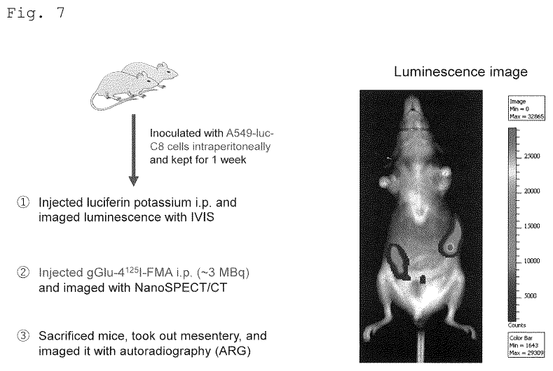

A model mouse was prepared in which A549 cells with high GGT activity were transplanted subcutaneously on the left side of the body, and H226 cells with low GGT activity were transplanted subcutaneously on the right side of the body. Into subcutaneous tumors of A549 (high GGT activity, left side) and H226 (low GGT activity, right side), gGlu-4¹²⁵I-FMA of about 150 kBq in 30 μL PBS(-) was directly administered. SPECT/CT images were acquired 30 minutes, 2 hours, and 5 hours after administration. The results are shown in FIG. 6.

From FIG. 6, it can be seen that the radioactivity is rapidly attenuated in the tumor with low GGT activity (H226 tumor), but the radioactivity clearly remains in the tumor with high GGT activity (A549 tumor). This result indicates that when gGlu-4¹²⁵I-FMA was applied in a local environment, a metabolic trap utilizing the activity of GGT was achieved in vivo.

### Example 4

Visualization experiment of peritoneal metastasis by intraperitoneal administration (i.p.) of probe

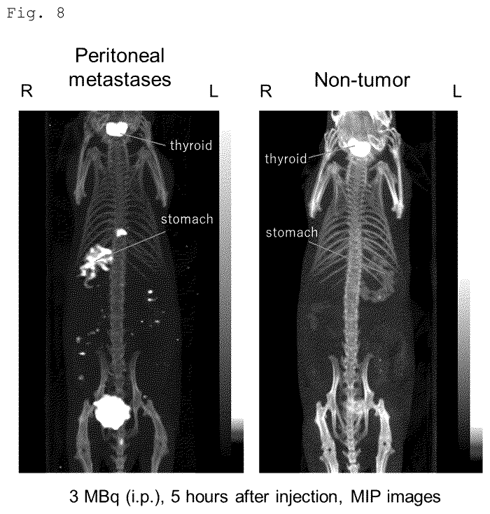

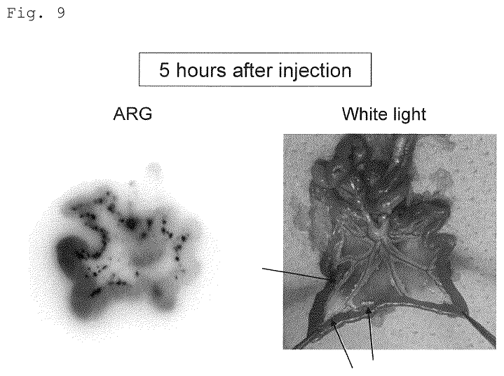

Next, according to the procedure shown in the left figure of FIG. 7, peritoneal dissemination model mice were prepared, and gGlu-4¹²⁵I-FMA was intraperitoneally administered. First, A549-luc-C8 cells with high GGT activity were transplanted into the abdominal cavity of the mouse and bred for 1 week. Luciferin potassium was intraperitoneally administered, and tumor growth was confirmed by luminescence imaging using IVIS (right figure in FIG. 7). Next, gGlu-4¹²⁵I-FMA was intraperitoneally administered (to 3 MBq) to the peritoneal model mouse and a tumor-free mouse, and SPECT/CT imaging was performed by NanoSPECT/CT. The imaging results at 5 hours after administration are shown in FIG. 8. The left figure shows the peritoneal dissemination model mouse, and the right figure shows the tumor-free mouse. They are shown as Maximum Intensity Projection (MIP) images.

From the SPECT/CT images, many signals gathered in the bladder after 30 minutes, and the probe almost disappeared from the abdominal cavity, but it can be said that this rate of excretion is a major feature of the present probe. From FIG. 8, at 5 hours after administration, only scattered signals remained in the abdomen in the peritoneal dissemination model mouse, and such a signal was not observed in the tumor-free mouse.

In addition, the peritoneal dissemination model mouse in the left figure was dissected after 5 hours of imaging, and the intestines and mesentery were taken out and imaged by autoradiography (ARG) (FIG. 9). An ARG image is shown in the left figure, and a white light image is shown in the right figure, and uptake of radionuclides was observed in agreement with minute peritoneal dissemination scattered in the intestines and mesentery as indicated by some arrows. From this, it is considered that the signals scattered in the abdominal cavity in the SPECT/CT on the left of FIG. 8 correspond to the peritoneal dissemination.

This result suggested that while the probe developed this time strongly accumulates in a tumor, the unreacted probe has a property of being rapidly excreted from the abdominal cavity, and thus there is a possibility that peritoneal disseminated lesions can be visualized with high contrast.

1. A compound represented by the following general formula (I) or a salt thereof. 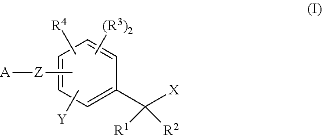
wherein:
X is selected from the group consisting of a fluorine atom, an ester group (—OC(═O)—R′), a carbonate group (—OCO₂—R′), a carbamate group (—OCONH—R′), phosphoric acid and its ester group (—OP(═O)(—OR′)(—OR″), and sulfuric acid and its ester group (—OSO₂—OR′),
wherein R′, R″ are each independently selected from a substituted or unsubstituted alkyl group or a substituted or unsubstituted aryl group;
Y is —NH—CO-L, —NH-L′, or —OL′,
wherein L is a partial structure of an amino acid,
L′ is a saccharide or a partial structure of a saccharide, a saccharide or a partial structure of a saccharide having a self-cleaving linker, or an amino acid or a peptide having a self-cleaving linker;
R¹ and R² are each independently selected from a hydrogen atom or a monovalent substituent;
R³ is a hydrogen atom or 1 to 2 identical or different monovalent substituents present on the benzene ring;
R⁴ is a hydrogen atom, or a substituent or molecule capable of altering pharmacokinetics, the substituent or molecule may be bonded to the benzene ring via a linker;
Z represents a single bond or a linking group; and
A represents a radionuclide.

2. The compound or a salt thereof according to claim 1, wherein the radionuclide is selected from the group consisting of ¹²⁵I, ²¹¹At, ¹⁸F, ¹⁵O, ¹²³I, ¹³¹I, ¹²⁴I, and ¹¹C.

3. The compound or a salt thereof according to claim 1, wherein the substituent or molecule capable of altering pharmacokinetics is introduced into the benzene ring via a linker or directly.

4. The compound or a salt thereof according to claim 1, wherein
the linker is selected from the group consisting of an alkylene group (with the proviso that one or more —CH₂— of the alkylene group may be replaced by —O—, —S—, —NH—, or —CO—), arylene (including heteroarylene), cycloalkylene, an alkoxyl group, a polyethylene glycol chain, and a group formed by arbitrarily bonding two or more groups selected from these groups.

5. The compound or a salt thereof according to claim 1, wherein the linking group of Z is selected from the group consisting of an alkylene group (with the proviso that one or more —CH₂— of the alkylene group may be replaced by —O—, —S—, —NH—, or —CO—), arylene (including heteroarylene), cycloalkylene, an alkoxyl group, a polyethylene glycol chain, and a group formed by arbitrarily bonding two or more groups selected from these groups.

6. The compound or a salt thereof according to claim 1, wherein the partial structure of an amino acid of L, together with C═O to which L is bonded, constitutes an amino acid, an amino acid residue, a peptide, or a part of an amino acid.

7. The compound or a salt thereof according to claim 1, wherein the partial structure of a saccharide of L′, together with O to which L′ is bonded, constitutes a saccharide, a part of a saccharide.

8. The compound or a salt thereof according to claim 1, wherein —Y in the general formula (I) is bonded to the ortho or para position of the benzene ring with respect to —C(R¹)(R²) X.

9. The compound or a salt thereof according to claim 1, wherein Y has a structure selected from the following.

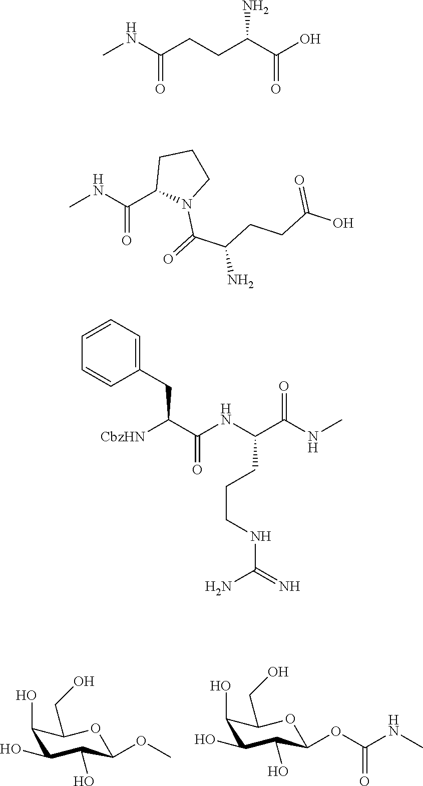

10. The compound or a salt thereof according to claim 1, wherein X is a fluorine atom or an ester group (—OCO—R′).

11. The compound or a salt thereof according to claim 1, wherein R¹ and R² are each independently selected from a hydrogen atom and a fluorine atom.

12. The compound or a salt thereof according to claim 1, wherein the monovalent substituent of R³ is selected from the group consisting of an alkyl group, an alkoxycarbonyl group (—CO—OR^{a}), a nitro group, an amino group, a hydroxyl group, an alkylamino group (—NHR^{a}, —NR^{a}₂), an alkoxy group (—OR^{a}), an ester group (—O—CO—R^{a}), a halogen atom, a boryl group, and a cyano group (R^{a} is a substituted or unsubstituted alkyl group or a substituted or unsubstituted aryl group, and when there are two or more R^{a}s, each R^{a} may be the same or different).

13. The compound or a salt thereof according to claim 12, wherein the monovalent substituent of R³ is an alkyl group or an alkoxycarbonyl group.

14. The compound or a salt thereof according to claim 12, wherein the monovalent substituent of R³ is a halogen atom.

15. The compound or a salt thereof according to claim 12, wherein at least one of the monovalent substituents of R³ is an alkyl group or an alkoxycarbonyl group, and at least one of the monovalent substituents of R³ is a halogen atom.

16. The compound or a salt thereof according to claim 1, wherein both R³ and R⁴ are a hydrogen atom.

17. A pharmaceutical composition comprising a compound according to claim 1 or a pharmaceutically acceptable salt thereof.

18. The pharmaceutical composition according to claim 17, which is used in nuclear medical examination.

19. The pharmaceutical composition according to claim 18, that acts cell-selectively by cancer cell-specific enzyme activity to be accumulated in cancer cells.

20. The pharmaceutical composition according to claim 19, wherein the enzyme is a peptidase or a glycosidase.

21. The pharmaceutical composition according to claim 18, wherein the nuclear medical examination is at least one selected from the group consisting of scintigraphy, SPECT (single-photon emission computed tomography), and PET (positron emission tomography).

22. A method for diagnosing a disease or a condition that may lead to a disease, the method comprising:
(A) administering to a subject having or suspected of having a disease or condition a medicament comprising the compound according to claim 1 or a pharmaceutically acceptable salt thereof; and
(B) examining the presence of one or more selected from the group consisting of a tumor, a cancer cell, and a cancer tissue in a target tissue or target organ of the subject by measuring radiation emitted from a radionuclide contained in the medicament localized in the target tissue or target organ.

23. The diagnostic method according to claim 22, wherein the medicament is administered to the subject intravenously, intraperitoneally, or intratumorally.

24. A kit comprising the compound of claim 1 or a pharmaceutically acceptable salt thereof.
